(19) United States
(12) Patent Application Publication
(10) Pub. No.: US 2003/0092636 A1
(43) Pub. Date: May 15, 2003

(54) Treatment and prevention of paresthesia comprising co-therapy with anticonvulsant derivatives and potassium

(76) Inventor: Stephen D. Silberstein, Philadelphia, PA (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/287,152

(22) Filed: Nov. 4, 2002

Related U.S. Application Data
(60) Provisional application No. 60/332,770, filed on Nov. 6, 2001.

(51) Int. Cl. A61K 31/7024; A61K 31/35; A61K 31/255; A61K 31/36

(52) U.S. Cl. 514/23; 514/459; 514/464; 514/517

(57) ABSTRACT

Co-therapy for the treatment and/or prevention of paraesthesia and/or dysesthesia comprising administering to a subject in need thereof a therapeutically effective amount of one or more anticonvulsant derivatives with potassium supplements.

### CROSS REFERENCE TO RELATED APPLICATION

This application claims the benefit of U.S. Provisional Application No. 60/332,770, filed on Nov. 06, 2001, which is incorporated by reference herein in its entirety.

### BACKGROUND OF THE INVENTION

The present invention is directed to co-therapy for the treatment and/or prevention of paresthesia and dysesthesia comprising administering to a subject in need thereof a therapeutically effective amount of anticonvulsant derivatives and potassium supplements.

Compounds of formula I:

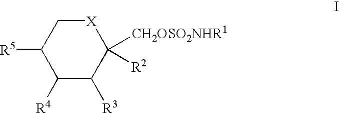

are structurally novel antiepileptic compounds that are highly effective anticonvulsants in animal tests (MARYANOFF, B. E, NORTEY, S. O., GARDOCKI, J. F., SHANK, R. P. AND DODGSON, S. P. J. Med. Chem. 1987, 30, 880-887; MARYANOFF, B. E., COSTANZO, M. J., SHANK, R. P., SCHUPSKY, J. J., ORTEGON, M. E., AND VAUGHT J. L. Bioorg. Med. Chem. Lett. 1993, 3, 2653-2656; SHANK, R. P., GARDOCKI, J. F., VAUGHT, J. L., DAVIS, C. B., SCHUPSKY, J. J., RAFFA, R. B., DODGSON, S. J., NORTEY, S. O., MARYANOFF, B. E. Epilepsia 1994, 35, 450-460; MARYANOFF B E, COSTANZO M J, NORTEY S O, GRECO M N, SHANK R P, SCHUPSKY J J, ORTEGON M P, VAUGHT J L. J. Med. Chem. 1998, 41, 1315-1343). These compounds are covered by three U.S. Pat. No. 4,513,006, U.S. Pat. No. 5,242,942, and U.S. Pat. No. 5,384,327. One of these compounds 2,3:4,5-bis-O-(1-methylethylidene)-β-D-fructopyranose sulfamate, known as topiramate, has been demonstrated in clinical trials of human epilepsy to be effective as adjunctive therapy or as monotherapy in treating simple and complex partial seizures and secondarily generalized seizures (E. FAUGHT, B. J. WILDER, R. E. RAMSEY, R. A. REIFE, L D. KRAMER, G. W. PLEDGER, R. M. KARIM et. al., Epilepsia 1995, 36 (S4), 33; S. K. SACHDEO, R. C. SACHDEO, R. A. REIFE, P. LIM and G. PLEDGER, Epilepsia 1995, 36 (S4), 33; T. A. GLAUSER, Epilepsia 1999, 40 (S5), S71-80; R. C. SACHDEO, Clin. Pharmacokinet. 1998, 34, 335-346), and is currently marketed for the treatment of seizures in patients with simple and complex partial epilepsy and seizures in patients with primary or secondary generalized seizures in the United States, Europe and most other markets throughout the world.

Compounds of formula I were initially found to possess anticonvulsant activity in the traditional maximal electroshock seizure (MES) test in mice (SHANK, R. P., GARDOCKI, J. F., VAUGHT, J. L., DAVIS, C. B., SCHUPSKY, J. J., RAFFA, R. B., DODGSON, S. J., NORTEY, S. O., and MARYANOFF, B. E., Epilepsia 1994, 35, 450460). Subsequent studies revealed that Compounds of Formula I were also highly effective in the MES test in rats. Topiramate was also found to effectively block seizures in several rodent models of epilepsy (J. NAKAMURA, S. TAMURA, T. KANDA, A. ISHII, K. ISHIHARA, T. SERIKAWA, J. YAMADA, and M. SASA, Eur. J. Pharmacol. 1994, 254, 83-89), and in an animal model of kindled epilepsy (A. WAUQUIER and S. ZHOU, Epilepsy Res. 1996, 24, 73-77).

Paresthesia is defined as a positive sensory phenomena, more particularly as a tingling, crawling or burning sensation of the skin, without an apparent stimulus. Dysesthesias are unpleasant sensory phenomena associated with stimuli that are normally non-noxious. Painful paresthesias and dysesthesias are often associated with diabetic neuropathy, or may be the result of adverse side effects of pharmacological treatment of disease. They may also be associated with other neuropathies including, but not limited to, nutritional neuropathies, alcoholic neuropathy, carcinomatous neuropathy and amyloid neuropathy. They are also associated with other central nervous system diseases, which include but are not limited to thalamic stroke, spinal cord disease (e.g., multiple sclerosis, spinal cord trauma), with occlusive peripheral vascular disease, and with hemodialysis.

One of the adverse events or side effects noted in patients treated with topiramate for a variety of disorders, including epilepsy, migraine headaches and bipolar disorder is paresthesia. The severity of the paresthesia can range from mild to severe, and in the most severe cases may result in discontinuation of treatment. While other adverse events associated with topiramate tend to be most prominent immediately after initiation of therapy, and tend to remit over several weeks with continued topiramate administration, paresthesias are less likely to remit spontaneously, may persist for several months or be persistent.

In patients experiencing paresthesia as a side effect of pharmacological treatment, the paresthesia is typically left untreated if mild, and treated pharmacologically with tricyclic antidepressants or analgesics (including narcotic analgesics) if more moderate or severe. In the most severe cases of paresthesia, the patient will often discontinue the drug treatment.

Critchlow, A. S., et al., in British Medical Journal, 289, (1984), p.21 describe no effect on the incidence of paresthesia upon withdrawal of potassium supplements in patients being treated with acetazolamide for glaucoma. Rudolph, J., et al., in Medizinische Klinik, 94 (7), (1999), pp.391-394 describe a case study in which a patient suffering from painful hypokalemia associated with painful leg paresthesia was treated with intravenous potassium chloride in combination with spirocolactone, resulting in stabilization of serum potassium, disappearance of paresthesia and normalization of muscle function and ECG. Kulka, P. J., et al., in Der Anaesthesist, 48 (12), (1999), pp.896-898 and references therein describe case studies of the effect of inadvertent infusion of potassium chloride into an epidural catheter resulting in paresis (slight paralysis or weakness of a limb) and/or painful paraesthesias in the patient's extremities.

It has now been found that co-therapy comprising a therapeutically effective amount of one or more compounds of formula I and potassium supplements is useful in the prevention and/or treatment of paresthesia and dysesthesia.

### DISCLOSURE OF THE INVENTION

The present invention is directed to a method for the treatment and/or prevention of paresthesia and/or dysesthesia comprising co-therapy with a therapeutically effective amount of a potassium supplement and a compound of the following formula I:

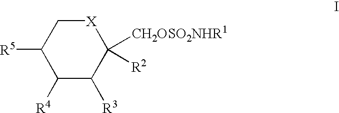

wherein X is O or CH₂, and R¹, R², R³, R⁴ and R⁵ are as defined hereinafter.

In an embodiment of the present invention is a method for decreasing the incidence and/or severity of paresthesia and/or dysesthesia comprising co-therapy with a therapeutically effective amount of a potassium supplement and a compound of the following formula I:

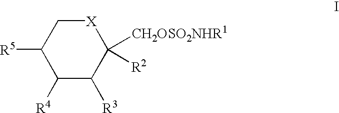

wherein X is O or CH₂, and R¹, R², R³, R⁴ and R⁵ are as defined hereinafter.

Illustrative of the invention is a pharmaceutical composition comprising a pharmaceutically acceptable carrier, one or more compounds of formula I and a potassium supplement. An illustration of the invention is a pharmaceutical composition made by mixing one or more compounds of formula I and a potassium supplement and a pharmaceutically acceptable carrier. Illustrating the invention is a process for making a pharmaceutical composition comprising mixing one or more compounds of formula I and a potassium supplement and a pharmaceutically acceptable carrier.

In another embodiment of the present invention is the use of any of the pharmaceutical compositions described above for the prevention of topiramate induced paresthesia or dysesthesia.

### DETAILED DESCRIPTION OF THE PREFERRED EMBODIMENTS

As used herein, the term “paresthesia” shall be defined as unpleasant spontaneous sensations, including, but not limited to, tingling, “pins and needles”, burning, pain, and electrical sensations, anywhere on the patient, more particularly in the extremities, i.e. arms and legs.

As used herein, the term “dysesthesia” shall be defined as unpleasant sensations evoked by normally non-noxious stimuli, including, but not limited to, tingling, “pins and needles”, burning, electrical sensations, and pain, anywhere on the patient, more particularly in the extremities, i.e. arms and legs.

As used herein, the term “potassium supplement” shall include any pharmaceutically acceptable source of potassium. Suitable examples include potassium chloride, potassium bicarbonate, potassium phosphate, and the like.

The term “prevention” means to anticipate and counter in advance. More particularly as in the present invention, prevention shall mean to prevent the onset of paresthesia or dysesthesia during treatment with topiramate, by initiating topiramate treatment simultaneously with potassium supplements.

As used herein, the term “subject”, refers to an animal, preferably a mammal, most preferably a human, who is the object of treatment, observation or experiment.

As used herein, the term “co-therapy” shall mean treatment of a subject in need thereof by administering one or more compounds of formula I with a potassium supplement, wherein the compound(s) of formula I and the potassium supplement are administered by any suitable means, simultaneously, sequentially, separately or in a single pharmaceutical formulation. Where the compound(s) of formula I and the potassium supplement are administered in separate dosage forms, the number of dosages administered per day for each compound may be the same or different. The compound(s) of formula I and the potassium supplement may be administered via the same or different routes of administration. Examples of suitable methods of administration are orally, intravenous (iv), intramuscular (im), and subcutaneous (sc). The active compounds may also be administrated directly to the nervous system including, but not limited to the intracerebral, intraventricular, intracerebroventricular, intrathecal, intracisternal, intraspinal and/or peri-spinal routes of administration by delivery via intracranial or intravertebral needles and/or catheters with or without pump devices. The compound(s) of formula I and the potassium supplement may be administered according to simultaneous or alternating regimens, at the same or different times during the course of the therapy, concurrently in divided or single forms.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. More particularly, in the present invention directed to co-therapy comprising administration of one or more compound(s) of formula I and a potassium supplements, “therapeutically effective amount” shall mean that amount of the combination of agents taken together so that the combined effect elicits the desired biological or medicinal response. For example, the therapeutically effective amount of co-therapy comprising administration of a compound of formula I and a potassium supplement would be the amount of the compound of formula I and the amount of the potassium supplement that when taken together or sequentially have a combined effect that is therapeutically effective. Further, it will be recognized by one skilled in the art that in the case of co-therapy with a therapeutically effective amount, as in the example above, the amount of the compound of formula I and/or the amount of the potassium supplement individually may or may not be therapeutically effective.

Optimal dosages and dosage regimens to be administered may be readily determined by those skilled in the art, and will vary with the mode of administration, the strength of the preparation and the advancement of the disease condition. In addition, factors associated with the particular patient being treated, including patient's sex, age, weight, diet, physical activity, time of administration and concomitant diseases, will result in the need to adjust dosages and/or regimens.

As used herein, the term “composition” is intended to encompass a product comprising the specified ingredients in the specified amounts, as well as any product which results, directly or indirectly, from combinations of the specified ingredients in the specified amounts.

To prepare the pharmaceutical compositions of this invention, one or more compounds of formula (I) are intimately admixed with a pharmaceutical carrier according to conventional pharmaceutical compounding techniques, which carrier may take a wide variety of forms depending on the form of preparation desired for administration, e.g., i.v. sterile injectable formulations will be prepared using appropriate solubilizing agents. A unit dose would contain about 15 to 200 mg of the active ingredient. The tablets contain some or all of the following inactive ingredients: lactose hydrous, pregelatinized starch, microcrystalline cellulose, sodium starch glycolate, magnesium stearate, purified water, carnauba wax, hydroxypropyl methylcellulose, titanium dioxide, polyethylene glycol, synthetic iron oxide, and polysorbate 80.

The sulfamates of the invention are compounds of the following formula I:

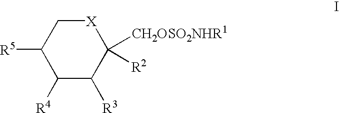

wherein

X is CH₂ or oxygen;

R¹ is hydrogen or alkyl; and

R², R³, R⁴ and R⁵ are independently hydrogen or lower alkyl and, when X is CH₂, R⁴ and R⁵ may be alkene groups joined to form a benzene ring and, when X is oxygen, R² and R³ and/or R⁴ and R⁵ together may be a methylenedioxy group of the following formula II:

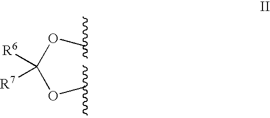

wherein

R⁶ and R⁷ are the same or different and are hydrogen, lower alkyl or are alkyl and are joined to form a cyclopentyl or cyclohexyl ring.

R¹ in particular is hydrogen or alkyl of about 1 to 4 carbons, such as methyl, ethyl and iso-propyl. Alkyl throughout this specification includes straight and branched chain alkyl. Alkyl groups for R², R³, R⁴, R⁵, R⁶ and R⁷ are of about 1 to 3 carbons and include methyl, ethyl, iso-propyl and n-propyl. When X is CH₂, R⁴ and R⁵ may combine to form a benzene ring fused to the 6-membered X-containing ring, i.e., R⁴ and R⁵ are defined by the alkatrienyl group ═C—CH═CH—CH═.

A particular group of compounds of formula I is that wherein X is oxygen and both R² and R³ and R⁴ and R⁵ together are methylenedioxy groups of the formula II, wherein R⁶ and R⁷ are both hydrogen, both alkyl or combine to form a spiro cyclopentyl or cyclohexyl ring, in particular where R⁶ and R⁷ are both alkyl such as methyl. A second group of compounds of formula I is that wherein X is CH₂ and R⁴ and R⁵ are joined to form a benzene ring. A third group of compounds of formula I is that wherein both R² and R³ are hydrogen.

The compounds of formula I may be synthesized by the following methods:

(a) Reaction of an alcohol of the formula RCH₂OH with a chlorosulfamate of the formula CISO₂NH₂ or CISO₂NHR¹ in the presence of a base such as potassium t-butoxide or sodium hydride at a temperature of about −20° to 250° C. and in a solvent such as toluene, THF, or dimethylformamide wherein R is a moiety of the following formula III:

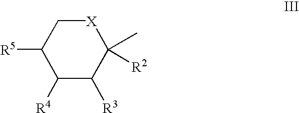

(b) Reaction of an alcohol of the formula RCH₂OH with sulfurylchloride of the formula SO₂Cl₂ in the presence of a base such as triethylamine or pyridine at a temperature of about −40° to 25° C. in a solvent such as diethyl ether or methylene chloride to produce a chlorosulfate of the formula RCH₂OSO₂Cl.

The chlorosulfate of the formula RCH₂OSO₂Cl may then be reacted with an amine of the formula R¹NH² at a temperature of about 40° to 25° C. in a solvent such as methylene chloride or acetonitrile to produce a compound of formula I. The reaction conditions for (b) are also described by T. Tsuchiya et al. in Tetrahedron Lett., 1978, 3365.

(c) Reaction of the chlorosulfate RCH₂OSO₂Cl with a metal azide such as sodium azide in a solvent such as methylene chloride or acetonitrile yields an azidosulfate of the formula RCH₂OSO₂N₃ as described by M. Hedayatullah in Tetrahedron Lett. 1975, 2455. The azidosulfate is then reduced to a compound of formula I wherein R¹ is hydrogen by catalytic hydrogenation, e.g. with a noble metal and H₂ or by heating with copper metal in a solvent such as methanol.

The starting materials of the formula RCH₂OH may be obtained commercially or as known in the art. For example, starting materials of the formula RCH₂OH wherein both R² and R³ and R⁴ and R⁵ are identical and are of the formula II may be obtained by the method of R. F. Brady in Carbohydr. Res. 1970, 14, 35 or by reaction of the trimethylsilyl enol ether of a R⁶COR⁷ ketone or aldehyde with fructose at a temperature of about 25° C., in a solvent such a halocarbon, e.g. methylene chloride in the presence of a protic acid such as hydrochloric acid or a Lewis Acid such as zinc chloride. The trimethylsilyl enol ether reaction is described by G. L. Larson et al. in J. Org. Chem. 1973, 38, 3935.

Further, carboxylic acids and aldehydes of the formulae RCOOH and RCHO may be reduced to compounds of the formula RCH₂OH by standard reduction techniques, e.g. reaction with lithium aluminum hydride, sodium borohydride or borane-THF complex in an inert solvent such a diglyme, THF or toluene at a temperature of about 0° to 100° C., e.g. as described by H. O. House in “Modern Synthetic Reactions”, 2nd Ed., pages 45 to 144 (1972).

The compounds of formula I may also be made by the process disclosed U.S. Pat. No. 4,513,006, U.S. Pat. No. 5,242,942, and U.S. Pat. No. 5,384,327, which are incorporated by reference herein.

The compounds of formula I include the various individual isomers as well as the racemates thereof, e.g., the various alpha and beta attachments, i.e., below and above the plane of the drawing, of R², R³, R⁴ and R⁵ on the 6-membered ring. Preferably, the oxygen of the methylenedioxy group of formula II are attached on the same side of the 6-membered ring.

The ability of co-therapy comprising administration of a therapeutically effective amount of one or more compounds of formula I and potassium supplements to treat or prevent paresthesia or dysesthesia is based on case studies described in more detail as follows.

### EXAMPLE 1

### Anecdotal Case Studies

Potassium chloride and food—orange juice and bananas—supplementation were effective in decreasing paresthesias and its severity. Patients complaining of persistent paresthesias were prescribed 20-40 meq/day of potassium chloride, which resulted in control of the paresthesias.

### EXAMPLE 2

### Open-Label Series Evaluating the Effect of Potassium Supplement on Parasthesias Induced by Topiramate

The objective of the study is to determine whether potassium supplementation may relieve or prevent paresthesias due to topiramate therapy in patients with migraine headache.

Overview of Study Design

This is a single center, outpatient, open-label, pilot study. Twenty to twenty five (20-25) patients receiving topiramate for migraine prophylaxis and who meet selection criteria as outlined below are enrolled in the study. In order to be enrolled, patients are required to experience acral paresthesias of at least moderate severity attributed to topiramate therapy. Upon enrollment, patients are prescribed oral potassium chloride supplementation, and the severity of paresthesias is re-evaluated at predetermined intervals as described below.

Inclusion/Exclusion Criteria for Enrollment

Inclusion Criteria:

1). Diagnosis of migraine headache;

2). Therapy with topiramate alone or in addition to other agents for prophylaxis of migraine;

3). Persistent paresthesias attributed to topiramate therapy, wherein the paresthesia is graded as moderate, severe, or excruciating;

4). Subject of either sex, between the ages of 18 to 70 years. If subject is a female of childbearing age, she must be (a) postmenopausal for at least one year, or (b) have had a hysterectomy, or tubal ligation, or otherwise be incapable of pregnancy, or (c) have practiced one of the following methods of contraception for at least one month prior to study entry: hormonal contraceptives, spermicide and barrier, intrauterine device, spousal/partner sterility, or (d) be practicing abstinence and agree to continue abstinence or to use an acceptable method of contraception (as listed above) should sexual activity commence. If (c) or (d), the subject must have had a negative pregnancy test (urine or serum) within one week of study entry.

Exclusion Criteria

1). Contraindications to topiramate therapy based on the precautions, warnings or contraindications in the topiramate package insert.

2). Medical contraindications to potassium chloride therapy.

3). Pregnant or lactating women.

4). Subjects taking carbonic anhydrase inhibitors.

Dosage and Administration

Potassium chloride is initiated at a dose of 16 milliequivalents per day, given as a BID dose, and titrated to clinical response over 1-2 weeks, in patients already receiving topiramate for prophylaxis of migraine headache.

Data Analysis

Efficacy is assessed by comparing severity of paresthesias prior to treatment with severity after initiation of potassium chloride supplementation at 2 days, one week, two weeks and one month (final visit). The paresthesia is graded on the following scale:

0—None

1—Mild

2—Moderate

3—Severe

4—Excruciating

Appropriate descriptive statistics are employed. Additional data is collected on patient demographics, coexistent neurologic and psychiatric diagnoses and concomitant medications.

Tolerability and safety is assessed by the evaluation of vital signs, physical examinations, evaluation of adverse events and, as appropriate, clinical laboratory tests. Pregnancy tests are performed prior to the administration of study medication and at the end of therapy for females of child bearing potential.

Adverse Events

All adverse events (AEs) are reported for a 24-hour period following administration of study medication. Serious adverse events (SAEs) occurring within a period of 30 days following the last intake of investigational product administration are also handled according to this procedure. Medical events that occur between the signing of the Informed Consent and the first intake of study drug are documented in the medical history section of the CRF and source document.

All AEs, regardless of seriousness, severity, or presumed relationship to study therapy, are recorded using medical terminology in the source document and on the CRF. Whenever possible, diagnoses are given when signs and symptoms are due to a common etiology (e.g., cough, runny nose, sneezing, sore throat, and head congestion should be reported as “upper respiratory infection”). Investigators record on the CRF their opinion concerning the relationship of the AE to study therapy. All measures required for AE management are recorded in the source document.

Subjects are encouraged to report treatment-emergent AEs spontaneously or in response to general, nondirected questioning (e.g., “How has your health been since the last visit”). For each treatment-emergent AE volunteered by the subject or noted by caregivers or clinician investigators, the investigator obtains all the information required to complete the AE page of the CRF.

### EXAMPLE 3

The patient was a 39-year-old woman who had cluster headaches. The patient was taking topiramate at 150 mg/day. She complained of paresthesias. The patient was given potassium chloride at 20 mEq/day. Within 9 days, it was observed that her paresthesias had abated.

### EXAMPLE 4

The patient was a 43-year-old woman who had migraine headaches and who was taking topiramate at 150 mg/day. She complained of paresthesias and was given potassium chloride at 20 mEq/day. After about one month, it was observed that her paresthesias had abated.

### EXAMPLE 5

The patient was a 54-year-old woman who had migraine headaches and was taking topiramate at 200 mg/day. She complained of paresthesias and was given potassium chloride at 20 mEq/day. After about two months it was observed that her paresthesias had abated.

### EXAMPLE 6

The patient was a 21-year-old woman who had migraine headaches and was taking topiramate at 125 mg/day. She complained of paresthesias and was given potassium chloride at 20 mEq/day. After about one month it was observed that her paresthesias had abated.

### EXAMPLE 7

The patient was a 59-year-old man who had migraine headaches and was taking topiramate at 50 mg/day. He complained of paresthesias and was given potassium chloride at 20 mEq/day. After one month it was observed that his paresthesias had abated.

Thus, for treating or preventing paresthesia or dysesthesia, one or more compounds of formula I may be administered as co-therapy in combination with potassium supplements. Preferably, the compound of formula I is topiramate. Preferably, the compound of formula I is administered at a dosage in the range of 10 to 650 mg/kg, more preferably at a dosage in the range of 25 to 325 mg/kg once or twice daily. Preferably, the potassium supplements are administered at a dosage in the range of about 10 meq/day to about 50 meq/day, more preferably, the potassium supplements are administered at a dosage in range of about 20 meq/day to about 40 meq/day.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

What is claimed is:

1. A method for treating paresthesia or dysesthesia in a subject in need thereof, comprising co-therapy with a therapeutically effective amount of a compound of the formula I: 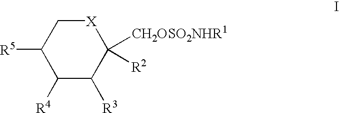
wherein
X is CH₂ or oxygen;
R¹ is hydrogen or alkyl; and
R², R³, R⁴ and R⁵ are independently hydrogen or lower alkyl and, when X is CH₂, R⁴ and R⁵ may be alkene groups joined to form a benzene ring and, when X is oxygen, R² and R³ and/or R⁴ and R⁵ together may be a methylenedioxy group of the following formula II: 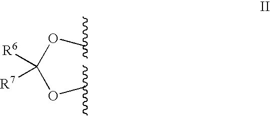
wherein
R⁶ and R⁷ are the same or different and are hydrogen, lower alkyl or are alkyl and are joined to form a cyclopentyl or cyclohexyl ring;
and a potassium supplement.

2. The method of claim 1 wherein the compound of formula I is topiramate.

3. The method of claim 2, wherein the amount of the compound of formula I is from about 10 to 650 mg daily.

4. The method of claim 3, wherein the amount of the compound of formula I is from about 25 to 325 mg once or twice daily.

5. The method of claim 1, wherein the amount of the potassium supplement is in the range of about 10 meq to about 50 meq daily.

6. The method of claim 5, wherein the amount of the potassium supplement is in the range of about 20 meq to about 40 meq daily.

7. A method for preventing paresthesia or dysesthesia in a subject in need thereof, comprising co-therapy with a therapeutically effective amount of a compound of the formula I: 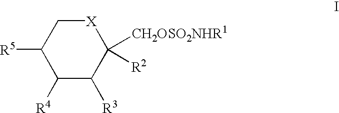
wherein
X is CH₂ or oxygen;
R¹ is hydrogen or alkyl; and
R², R³, R⁴ and R⁵ are independently hydrogen or lower alkyl and, when X is CH₂, R⁴ and R⁵ may be alkene groups joined to form a benzene ring and, when X is oxygen, R² and R³ and/or R⁴ and R⁵ together may be a methylenedioxy group of the following formula II: 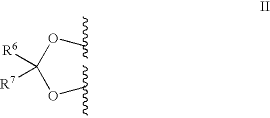
wherein
R⁶ and R⁷ are the same or different and are hydrogen, lower alkyl or are alkyl and are joined to form a cyclopentyl or cyclohexyl ring;
and a potassium supplement.

8. The method of claim 7 wherein the compound of formula I is topiramate.

9. The method of claim 8, wherein the amount of the compound of formula I is from about 10 to 650 mg daily.

10. The method of claim 9, wherein the amount of the compound of formula I is from about 25 to 325 mg once or twice daily.

11. The method of claim 7, wherein the amount of the potassium supplement is in the range of about 10 meq to about 50 meq daily.

12. The method of claim 11, wherein the amount of the potassium supplement is in the range of about 20 meq to about 40 meq daily.

13. A method for reducing the incidence or severity of paresthesia or dysesthesias in a subject in need thereof, comprising co-therapy with a therapeutically effective amount of a compound of the formula I: 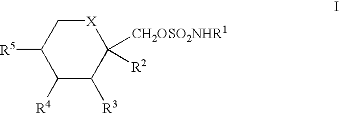
wherein
X is CH₂ or oxygen;
R¹ is hydrogen or alkyl; and
R², R³, R⁴ and R⁵ are independently hydrogen or lower alkyl and, when X is CH₂, R⁴ and R⁵ may be alkene groups joined to form a benzene ring and, when X is oxygen, R² and R³ and/or R⁴ and R⁵ together may be a methylenedioxy group of the following formula II: 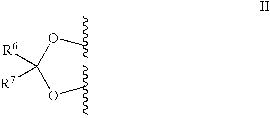
wherein
R⁶ and R⁷ are the same or different and are hydrogen, lower alkyl or are alkyl and are joined to form a cyclopentyl or cyclohexyl ring;
and a potassium supplement.

14. The method of claim 13 wherein the compound of formula I is topiramate.

15. The method of claim 14, wherein the amount of the compound of formula I is from about 10 to 650 mg daily.

16. The method of claim 15, wherein the amount of the compound of formula I is from about 25 to 325 mg once or twice daily.

17. The method of claim 13, wherein the amount of the potassium supplement is in the range of about 10 meq to about 50 meq daily.

18. The method of claim 17, wherein the amount of the potassium supplement is in the range of about 20 meq to about 40 meq daily.

19. A pharmaceutical composition comprising topiramate, a potassium supplement and a pharmaceutically acceptable carrier.

20. A pharmaceutical composition made by mixing topiramate, a potassium supplement and a pharmaceutically acceptable carrier.

21. A process for making a pharmaceutical composition comprising mixing topiramate, a potassium supplement and a pharmaceutically acceptable carrier.
